(19) United States
(12) Patent Application Publication
(10) Pub. No.: US 2015/0086676 A1
(43) Pub. Date: Mar. 26, 2015

(54) AQUEOUS, STERILE VITAMIN FORMULATION

(71) Applicant: DSM IP ASSETS B.V., Heerlen (NL)

(72) Inventor: Hendrik Ruijter, Basel (CH)

(21) Appl. No.: 14/396,649

(22) PCT Filed: May 2, 2013
(86) PCT No.: PCT/EP2013/059087

§ 371 Date: Oct. 23, 2014

(30) Foreign Application Priority Data
May 2, 2012 (EP) 12166411.4

(51) Int. Cl. A61K 31/375; A23L 2/02; A61K 31/51; A61K 31/4188; A61K 31/525; A61K 31/455; A61K 31/197; A23L 1/302; A61K 31/714

(52) U.S. Cl. CPC A61K 31/375; A23L 1/302; A23L 2/02; A61K 31/51; A61K 31/714; A61K 31/525; A61K 31/455; A61K 31/197; A61K 31/4188; A23V 2002/00
USPC 426/72; 514/52

(57) ABSTRACT

The present invention relates to a process for production of a sterile aqueous formulation comprising water-soluble vitamins. Such a formulation is useful as a premix for the production of food, feed and personal care products. Furthermore the invention is also related to such sterile aqueous formulations as well as the use of them in the production of food, feed or personal care products, which are pasteurized.

The present invention relates to a process for production of a sterile aqueous formulation comprising water-soluble vitamins. Such a formulation is useful as a premix for the production of food, feed and personal care products. Furthermore the invention is also related to such sterile aqueous formulations as well as the use of them in the production of food, feed or personal care products, which are pasteurized.

The aqueous and sterile vitamin formulations, obtained from the process as described and claimed herein are very stable (storage stable) and very useful as premixes for the preparation of feed, food or personal care products.

Vitamin enriched products are widely spread. Such products can be found in numerous fields of applications. The vitamins fulfil different purposes in the end-market product, such as antioxidative effects and/or health benefits.

The present invention relates to formulations comprising vitamin C and at least one other water soluble vitamin.

Water-soluble vitamins dissolve easily in water.

Important water-soluble vitamins are vitamin B₁ (thiamine), vitamin B₂ (riboflavin), vitamin B₃ (niacin, niacinamide), vitamin B₅ (pantothenic acid), vitamin B₆ (pyridoxine, pyridoxamine, pyridoxal), vitamin B₇ (biotin), vitamin B₉ (folic acid, folinic acid), vitamin B₁₂ (cyanocobalamin, hydroxycobalamin, methylcobalamin), and vitamin C (ascorbic acid).

All the end-market products (i.e. products, which are sold for example in grocery stores) must be storage-stable and they are very often (especially food products) pasteurized. During the necessary pasteurisation step there is always a loss of concentration of some of the ingredients (also of the water-soluble vitamins).

Due to that undesired side-effect, it is necessary to “overdose” the unpasteurised product, so that in the end-product the needed (and indicated) amount of the water-soluble vitamins is present.

The goal of the present invention was to find a way how to avoid the loss of vitamins in the production of pasteurised products, which allows to reducing the amount of water-soluble vitamins, which is used.

Surprisingly it was found that a well-defined aqueous and sterile formulation comprising water-soluble vitamins can be used as a premix, which is added to the food feed and/or personal product after pasteurisation.

Therefore the present invention relates to a process for production of a sterile aqueous formulation wherein the aqueous formulation comprises
(i) at least 20 g/l of vitamin C, and
(ii) at least 10 g/l of at least one further water-soluble vitamin, and
(iii) water, and wherein the process is characterized in that the aqueous formulation is filtered by using a filter with a pore size of ≧0.22 μm.

The aqueous formulation (=the premix) according to the present invention is sterile. This is done by using sterile filtration. Under the term sterile filtration in context of the present invention microfiltration using membrane filters is meant. The pore size of the membrane filters is usually 0.22 μm. Such membranes do effectively remove microorganisms, which makes the aqueous formulation sterile.

Membrane filters are commonly made from materials such as mixester cellulose or poly-ethersulfone (PES). The filtration equipment and the filters themselves are available commercially.

As a consequence of this filtration step the aqueous formulation only comprises particles which are smaller than 0.22 μm after the filtration.

The aqueous formulation according to the present invention comprises at least 20 g/l of vitamin C. Preferably it comprises 20 g/l to 50 g/l of vitamin C.

The aqueous formulation according to the present invention preferably comprises at least 30 g/l-60 g/l of at least one further water-soluble vitamin.

Therefore the present invention also relates to a process for production of a sterile aqueous formulation wherein the aqueous formulation comprises
(i) 20 g/l to 50 g/l of vitamin C, and
(ii) at least 30 g/l-60 g/l of at least one further water-soluble vitamin, and
(iii) water, and wherein the process is characterized in that the aqueous formulation is filtered by using a filter with a pore size of ≧0.22 μm.

The filtration step does not affect the concentration of the water-soluble vitamins. The concentration of the water-soluble vitamins is essentially the same before and after the filtration.

The sterile aqueous formulation is also storage stable for months.

The content of each water-soluble vitamin in the premix can depend on the product wherein the premix is incorporated.

The aqueous formulation of the present invention comprises water-soluble vitamins. The aqueous formulation always comprises 20 g/l of vitamin C and at least one further water-soluble vitamin.

Preferably the aqueous formulation according to the present invention comprises at least 20 g/l of vitamin C and at least 10 g/l of at least one water-soluble vitamin chosen from the group consisting of vitamin B₁, vitamin B₂, vitamin B₃, vitamin B₅, vitamin B₆, vitamin B₇, vitamin B₉ and vitamin B₁₂.

Therefore the present invention also relates to a process for production of a sterile aqueous formulation wherein the aqueous formulation comprises
(i) at least 20 g/l of vitamin C, and
(ii) at least 10 g/l of at least one water-soluble vitamin chosen from the group consisting of vitamin B₁, vitamin B₂, vitamin B₃, vitamin B₅, vitamin B₆, vitamin B₇, vitamin B₉ and vitamin B₁₂, and
(iii) water, and wherein the process is characterized in that the aqueous formulation is filtered by using a filter with a pore size of ≧0.22 mm.

An aqueous formulation according to the present invention can also comprise further ingredients. Such ingredients (=auxiliary agent) can be useful for the properties of the aqueous sterile formulation and/or for the properties of the food, feed and/or personal care products, wherein the aqueous sterile formulation is incorporated into.

Such auxiliary agents can be antioxidants, dyes, fat-soluble vitamins, fragrances, fillers (such as saccharose, maltodextrin) and buffers.

To ensure that the formulation is (or stays) sterile, the further ingredient(s) should be added to the sterile aqueous formulation before the filtration step, or if added afterwards the ingredient(s) must in a sterile form.

To maintain the sterility of the formulation, it is preferred that the sterile aqueous formulation as described above is filled and stored in a package which is capable to ensure the sterility.

It is clear that the sterile aqueous formulation as described above can be used in the production of a food, feed and/or personal care product just after it is produced. Storage is not needed in such a case and therefore no sterile packaging is needed.

Nevertheless to ensure that the final food, feed and personal care product is sterile (or fulfils the standards for the desired product) a suitable production environment has be provided.

Formulations obtained by the process are also part of the present invention. All the preferences described for the process apply as well.

Therefore the present invention also relates to a sterile aqueous formulation obtainable by a process wherein an aqueous formulation comprising
(i) 20 g/l to 50 g/l of vitamin C, and
(ii) at least 30 g/l-60 g/l of at least one further water-soluble vitamin, and
(iii) water,
   is filtered by using a filter with a pore size of ≧22 μm.

The sterile aqueous formulations as described above are also not known as such from the prior art. Therefore a further embodiment of the present invention are the aqueous sterile formulations as described above.

Therefore present invention also relates to a sterile aqueous formulation (I) wherein the formulation comprises
(i) at least 20 g/l of vitamin C, and
(ii) at least 10 g/l of at least one further water-soluble vitamin, and
(iii) water, and wherein
   the formulation only comprises particles with a particle size of less than 0.22 μm.

Furthermore the invention also relates to a sterile aqueous formulation (II), which is a formulation (I), which comprises 20 g/l to 50 g/l of vitamin C.

Furthermore the invention also relates to a sterile aqueous formulation (III), which is a formulation (I) and/or (II), which comprises
(i) at least 10 g/l of at least one water-soluble vitamin chosen from the group consisting of vitamin B₁, vitamin B₂, vitamin B₃, vitamin B₅, vitamin B₆, vitamin B₇, vitamin B₉ and vitamin B₁₂, and
(ii) at least 20 g/l of vitamin C.

Furthermore the invention also relates to a sterile aqueous formulation (IV), which is a formulation (I), (II) and/or (III) which comprises at least one auxiliary agent.

The formulations (I), (II), (III) and (IV) are used as premixes. Such premixes are used to be incorporated into food, feed and/or personal care product.

Due to the properties of these sterile aqueous formulations (storage stable, sterile) they are added (incorporated into) to the product after the pasteurization step.

Therefore the present invention also relates to the use of formulation (I), (II), (III) and/or (IV) in the process of production of pasteurized food, feed and/or personal care product, characterized in that the formulation (I), (II), (III) and/or (IV) is added to the product after pasteurization.

The production process can vary depending on the food, feed and/or personal care product which is produced.

The essential feature is that the formulations (I), (II), (III) and/or (IV) are always added after pasteurization. It is possible that further steps are carried out after the adding of formulation (I), (II), (III) and/or (IV).

It is also possible that more than one pasteurization step is needed and in such processes the formulations (I), (II), (III) and/or (IV) are always added after the last pasteurization step.

Preferably formulations (I), (II), (III) and/or (IV) are incorporated into liquid or gel-like product forms.

Such liquid products can be pasteurized food, feed or personal products. Any product can be produced, which needs to contain water-soluble vitamins and which need to be pasteurized.

Preferred pasteurized products are drinks (such as fruit juices), dairy products (such as yogurts, milk drinks).

Therefore the present invention also relates to pasteurized food, feed or personal products obtainable by producing the food, feed or personal product, wherein this process at least one pasteurisation step is carried out and wherein after the last pasteurisation step formulation (I), (II), (III) and/or (IV) is added.

### EXAMPLES

### Example 1

An aqueous formulation was produced by adding 1430 mg of vitamin B₁, 1705 mg of vitamin B₂, 1480 mg of vitamin B₃, 5760 mg of vitamin B₅, 2070 mg of vitamin B₆, 125 mg of vitamin B₇, 850 mg of vitamin B₁₂ and 22520 mg of vitamin C to 1 l of demineralised water.

The solution was stirred until a homogenous solution was obtained. Afterwards the solution was filtered by using a filter with a membrane size of 0.4 m.

A sterile aqueous formulation was obtained.

The formulation was packed in and stored at room temperature for 6 months.

After 6 months, the sterile aqueous formulation had the following content of the water soluble vitamins

1368 mg of vitamin B₁, 1533 mg of vitamin B₂, 1201 mg of vitamin B₃, 5170 mg of vitamin B₅, 2034 mg of vitamin B₆, 116 mg of vitamin B₇, 444 mg of vitamin B₁₂ and 19930 mg of vitamin C.

### Example 2

An aqueous formulation was produced by first mixing the following water-soluble vitamins (in dry form)

24 g of vitamin B₁, 36.3 g of vitamin B₂, 21.6 g of vitamin B₃, 66.6 g of vitamin B₅, 34.1 g of vitamin B₆, 168.6 g of vitamin B₇, 12 g of vitamin B₁₂ and 533 g of vitamin C to 1 l of water.

Additionally 168.66 g of saccharose was added.

62.5 g of that dry mixture was solved in 1 l of demineralised water and afterwards filtered through a membrane filter with a pore size of 0.2 μm.

A sterile aqueous formulation was obtained.

2.0 g of this sterile aqueous solution was added to a pasteurized fruit juice (orange juice).

Commercially available orange juice base was used for that purpose.

### Example 3

The same procedure as in example 2 was used to produce a pasteurized orange juice having the following content of water-soluble vitamins

2.51 mg/l of vitamin B₁, 1.75 mg/l of vitamin B₂, 2.80 mg/l of vitamin B₃, 8.9 mg/l of vitamin B₅, 3.37 mg/l of vitamin B₆, 240 μg/l of vitamin B₇, 1.90 μg/l of vitamin B₁₂ and 295 mg/l of vitamin C.

The juice was stored for 1 month (in a juice container) and the content of the water-soluble vitamins was measured again:

2.40 mg/l of vitamin B₁, 1.80 mg/l of vitamin B₂, 2.75 mg/l of vitamin B₃, 10.9 mg/l of vitamin B₅, 3.21 mg/l of vitamin B₆, 200 μg/l of vitamin B₇, 1.890 μg/l of vitamin B₁₂ and 283 mg/l of vitamin C.

It can be seen that an orange juice comprising a formulation according to the present invention is very stable.

1. A process for production of a sterile aqueous formulation wherein the aqueous formulation comprises
(i) at least 20 g/l of vitamin C, and
(ii) at least 10 g/l of at least one further water-soluble vitamin, and
(iii) water, and wherein
the process is characterized in that the aqueous formulation is filtered by using a filter with a pore size of ≧0.22 μm.

2. Process according to claim 1, wherein the aqueous formulation comprises 20 g/l to 50 g/l of vitamin C.

3. Process according to claim 1, wherein the aqueous formulation comprises at least 30 g/l-60 g/l of at least one further water-soluble vitamin.

4. Process according to claim 1, wherein the aqueous formulation comprises at least 20 g/l of vitamin C and at least 10 g/l of at least one water-soluble vitamin chosen from the group consisting of vitamin B₁, vitamin B₂, vitamin B₃, vitamin B₅, vitamin B₆, vitamin B₇, vitamin B₉ and vitamin B₁₂.

5. Process according to claim 1, wherein the aqueous formulation comprises further ingredients.

6. A sterile aqueous formulation wherein the formulation comprises
(i) at least 20 g/l of vitamin C, and
(ii) at least 10 g/l of at least one further water-soluble vitamin, and
(iii) water, and wherein
the formulation only comprises particles with a particle size of less than 0.22 μm.

7. A formulation according to claim 6, wherein the formulation comprises 20 g/l to 50 g/l of vitamin C.

8. A formulation according to claim 6, wherein the formulation comprises at least 10 g/l of at least one water-soluble vitamin chosen from the group consisting of vitamin B₁, vitamin B₂, vitamin B₃, vitamin B₅, vitamin B₆, vitamin B₇, vitamin B₉ and vitamin B₁₂, and at least 20 g/l of vitamin C.

9. A formulation according to claim 6, wherein the formulation comprises at least one auxiliary agent.

10. Use of a formulation of claim 6 in the process of production of pasteurized food, feed and/or personal care product wherein the formulation is added to the product after pasteurization.

11. Pasteurized food, feed or personal products comprising a formulation of claim 6.
